(19) United States
(12) Patent Application Publication
(10) Pub. No.: US 2025/0194632 A1
(43) Pub. Date: Jun. 19, 2025

(54) METHOD FOR PRODUCING HIGH-PURITY RICE PROTEIN POWDER AND RICE SYRUP

(71) Applicant: WUHAN MEI WEI YUAN BIOLOGICAL ENGINEERING CO., LTD., Wuhan, Hubei (CN)

(72) Inventors: Chuanjun Zou, Wuhan, Hubei (CN); Jiarui Zou, Wuhan, Hubei (CN)

(21) Appl. No.: 18/845,898

(22) PCT Filed: Mar. 15, 2023
(86) PCT No.: PCT/CN2023/081502

§ 371 Date: Sep. 11, 2024

(30) Foreign Application Priority Data
Mar. 18, 2022 (CN) 202210269760.7

(51) Int. Cl. A23J 1/12; A23L 7/104; A23L 7/25; B01D 21/26; B01D 25/12

(52) U.S. Cl. CPC A23J 1/125; A23L 7/107; A23L 7/25; B01D 21/262; B01D 25/12

(57) ABSTRACT

A method for producing a high-purity rice protein powder and a rice syrup is provided. The method includes: homogenizing a rice slurry, adding amylase thereto, heating the resulting mixture for a reaction to obtain a rice syrup liquid, and subjecting same to centrifugal separation to separately obtain a liquid-phase rice sugar liquid and a first precipitate; subjecting the liquid-phase rice sugar liquid to decoloration, ion exchange and evaporation to obtain a rice syrup, adding the first precipitate into second wastewater, and subjecting same to washing and centrifugal dewatering to obtain a second precipitate; adding pure water into the second precipitate, stirring same until uniform, heating same to 60° C. or more, maintaining the temperature, and dehydrating same to obtain a filter cake and second wastewater; and crushing and drying the filter cake to obtain a rice protein powder with a high purity and without thermal denaturation.

### FIELD OF THE INVENTION

The present disclosure relates to the technical field of deep processing of rice, and more particularly to a method for producing a high-purity rice protein powder and a rice syrup.

### BACKGROUND OF THE INVENTION

Vegetable protein powder refers to a powdered substance formed from proteins extracted from plants such as grains and beans by processing such as crushing, purifying and drying.

Compared with other plant starch granules, rice starch granules are very small, 2-8 μm, and uniform in particle size. Simultaneously producing a rice syrup and a protein powder from rice as a raw material mainly adopts an alkali-solution and acid-isolation method, rice protein is mainly alkali-soluble glutenin, and the protein is dissolved with an alkali, and then filtering is performed to obtain starch, and then an acid is added into the dissolved protein to obtain a protein precipitate, and the protein requires high temperature sterilization and spray drying, which is the most mature method at present and is also a method that is actually used in production.

Other methods include a protein residue extraction method, which involves obtaining a high-temperature denatured protein residue with a protein content of 40-70% during the production of a rice syrup, and further enzymatic hydrolysis and defatting to obtain rice protein with a purity of 80% or more.

### DEFECTS IN THE PRIOR ART

the alkali-solution and acid-isolation method needs to consume a large amount of alkali and produces a large amount of wastewater. The alkali will destroy the structure of protein and starch, and cause many harmful side reactions. High temperature sterilization and spray drying before protein drying will also lead to protein denaturation.

An enzymatic process is mild, but the production efficiency is low, and starch and protein cannot be obtained at the same time.

The protein residue extraction method is complicated in process and high in cost, heat denaturation of proteins is severe, and many impurities are introduced during enzymatic hydrolysis.

A solvent method has low efficiency and complex process, making it unsuitable for industrialization.

Therefore, it is necessary to provide a method for producing a high-purity rice protein powder and a rice syrup.

### SUMMARY OF THE INVENTION

In view of the problems existing in the prior art, the present disclosure proposes a method for producing a high-purity rice protein powder and a rice syrup, which combines physical extraction with enzymatic extraction to achieve the purposes of environmental friendliness in the extraction process, improving the quality of the rice protein powder and reducing the production cost.

In some embodiments of the present disclosure, disclosed is a method for producing a high-purity rice protein powder and a rice syrup, including the steps of:
S1, crushing rice, adding water for blending to obtain a first slurry, and homogenizing the first slurry with a high pressure homogenizer to obtain a second slurry;
S2, adding amylase into the second slurry, heating the resulting mixture, and performing heat preservation so that the second slurry sufficiently reacts with the amylase to obtain a raw rice syrup liquid;
S3, subjecting the raw rice syrup liquid to centrifugal separation to separately obtain a liquid-phase rice sugar liquid and a first precipitate;
S4, sequentially subjecting the liquid-phase rice sugar liquid obtained in S3 to decoloration, ion exchange, and evaporation to obtain the rice syrup;
S5, washing the first precipitate obtained in S3 with water, and performing centrifugal dewatering to obtain a second precipitate;
S6, adding pure water into the second precipitate, performing uniform stirring, and performing heating and heat preservation to obtain a solid-liquid mixed material;
S7, subjecting the material to filtration and dewatering to obtain a filter cake; and
S8, crushing and drying the filter cake to obtain the high-purity rice protein powder.

In some embodiments of the present disclosure, in S5, first wastewater is generated during the washing and centrifugal dewatering of the first precipitate, the first wastewater is recycled, and the recycled first wastewater is applied to the process of blending the first slurry in S1.

In some embodiments of the present disclosure, in S7, second wastewater is generated during the filtration and dewatering of the material, the second wastewater is recycled, and the recycled second wastewater is applied to the process of washing the first precipitate with the water in S5.

In some embodiments of the present disclosure, in S1, a mass ratio of the rice to the water in the first slurry is 1:1.5.

In some embodiments of the present disclosure, in S1, the first slurry is homogenized under a homogenization pressure of greater than 15 MPa at a temperature of not more than 55° C.

In some embodiments of the present disclosure, in S2, the amylase is alpha-amylase;
the second slurry is heated at a temperature in the range of 50-110° C.;
a duration of the heat preservation during the reaction between the second slurry and the amylase is in the range of 60-120 min.

In some embodiments of the present disclosure, the centrifugal separation in S3 uses a horizontal spiral centrifuge.

In some embodiments of the present disclosure, in S5, a ratio of a mass of the first precipitate to a mass of the water added during the washing of the first precipitate ranges from 1:3 to 1:10.

In some embodiments of the present disclosure, in S6, a ratio of a mass of the second precipitate to a mass of the pure water added to the second precipitate ranges from 1:3 to 1:10; and
the heating temperature is kept to be 80° C. or more, and the heat preservation is performed for 10 min or more.

In some embodiments of the present disclosure, in S7, the material is filtered by using a diaphragm plate and frame filter press to obtain the filter cake.

In some embodiments of the present disclosure, in S8, a microwave vacuum drying device is employed.

In some embodiments of the present disclosure, in S4, the liquid-phase rice sugar liquid can further be subjected to saccharification, activated carbon decoloration, ion exchange, evaporation and concentration to obtain the rice syrup.

The beneficial effects of the present disclosure are as follows:
the high-purity rice protein powder and rice syrup are produced by a physical method combined with an enzyme method, and there is no harmful chemical addition of foreign matters in the whole process, thus ensuring the safety of the product. The protein is purified, dried and sterilized under mild conditions, the extraction rate is high, the process is simple, the protein is not denatured, the production cost is low, and the product quality is good.

The entire process according to the present disclosure also solves the problem of treating wastewater generated during the washing process in the process. The wastewater is recycled multiple times, so that the discharge amount of the wastewater is low, which not only reduces the production cost, but also eliminates waste of resources.

### BRIEF DESCRIPTION OF DRAWINGS

FIG. 1 is a flow diagram of a process for producing a high-purity rice protein powder and a rice syrup according to an example of the present disclosure.

### DETAILED DESCRIPTION OF THE INVENTION

The technical solutions in the examples of the present disclosure will be clearly and completely described below in conjunction with the examples of the present disclosure. Obviously, the described examples are only a part of the examples of the present disclosure, rather than all of the examples. Based on the examples in the present disclosure, all other examples obtained by those of ordinary skill in the art without making inventive steps belong to the scope of protection of the present disclosure.

It should be noted that in the present disclosure, unless otherwise specified, all the embodiments and preferred embodiments mentioned herein can be combined with each other to form new technical solutions. In the present disclosure, unless otherwise specified, all the technical features and preferred features mentioned herein can be combined with each other to form new technical solutions. In the present disclosure, unless otherwise specified, percentages (%) or parts refer to weight percentages or weight parts relative to a composition. In the present disclosure, unless otherwise specified, the components referred to or preferred components thereof can be combined with each other to form new technical solutions. In the present disclosure, unless otherwise specified, a numerical range “a-b” represents an abbreviated representation of any combination of real numbers between a and b, wherein a and b are both real numbers. For example, a numerical range of “6-22” indicates that all real numbers between “6-22” have been listed herein in their entirety, with “6-22” being merely an abbreviated representation of combinations of these numerical values. A “range” disclosed herein may be one or more lower limits and one or more upper limits in the form of a lower limit and an upper limit. In the present disclosure, unless otherwise specified, each reaction or operation step may be performed sequentially or in sequence. Preferably, the reaction process herein is carried out sequentially.

Unless otherwise specified, the technical and scientific terms used herein have the same meanings as those familiar to those skilled in the art. In addition, any method or material similar or equivalent to the contents described can also be applied to the present disclosure.

The test materials used in the following examples, unless otherwise specified, were purchased from conventional biochemical reagent stores.

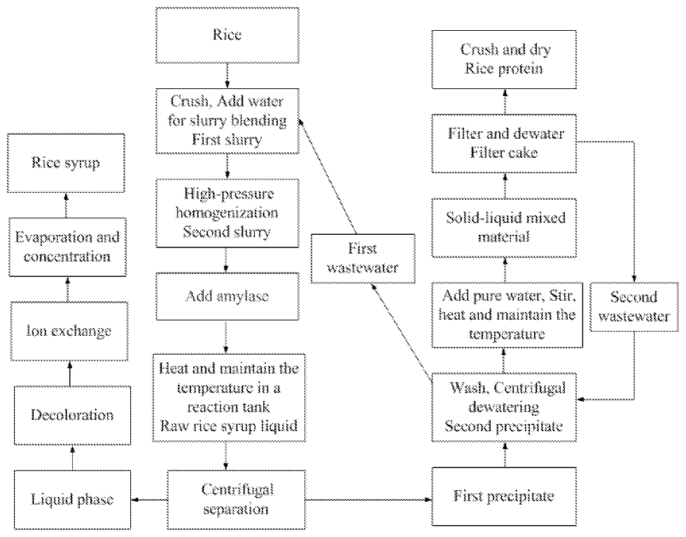

As shown in FIG. 1, a method for producing a high-purity rice protein powder and a rice syrup includes the steps of:
S1, crushing rice, adding water for blending to obtain a first slurry, and homogenizing the first slurry with a high pressure homogenizer to obtain a second slurry;
S2, adding amylase into the second slurry, heating the resulting mixture, and performing heat preservation so that the second slurry sufficiently reacts with the amylase to obtain a raw rice syrup liquid;
S3, subjecting the raw rice syrup liquid to centrifugal separation to separately obtain a liquid-phase rice sugar liquid and a first precipitate;
S4, sequentially subjecting the liquid-phase rice sugar liquid obtained in S3 to decoloration, ion exchange, and evaporation to obtain the rice syrup;
S5, washing the first precipitate obtained in S3 with water, and performing centrifugal dewatering to obtain a second precipitate;
S6, adding pure water into the second precipitate, performing uniform stirring, and performing heating and heat preservation to obtain a solid-liquid mixed material;
S7, subjecting the material to filtration and dewatering to obtain a filter cake; and
S8, crushing and drying the filter cake to obtain the high-purity rice protein powder.

In some examples of the present disclosure, in S1, a mass ratio of the rice to the water in the first slurry is 1:1.5.

On the basis of the above examples, the rice includes polished long-grained non-glutinous rice, polished round-grained rice, and glutinous rice.

In some examples of the present disclosure, in S1, the first slurry is homogenized under a homogenization pressure of greater than 15 MPa at a temperature of not more than 55° C.

It should be noted that components of a rice slurry are crushed by the high pressure homogenizer, and then physical centrifugation is performed by a centrifuge to separate out protein and starch. The production technique adopts all physical technical methods, which does not change the nature of the protein, is conducive to maintaining the characteristics and taste of the protein, and avoids starch gelatinization and protein denaturation.

In some examples of the present disclosure, in S2, the amylase is alpha-amylase;
the second slurry is heated at a temperature in the range of 50-110° C.; and
a duration of the heat preservation during the reaction between the second slurry and the amylase is in the range of 60-120 min.

In some examples of the present disclosure, in both the centrifugal separation process in S3 and the centrifugal dewatering process in S5, a centrifugal device uses a horizontal spiral centrifuge.

In some examples of the present disclosure, in S4, the liquid-phase rice sugar liquid can further be subjected to saccharification, activated carbon decoloration, ion exchange, evaporation and concentration to obtain the rice syrup.

In some examples of the present disclosure, in S8, a drying device used is a microwave vacuum drying device, which can play a sterilizing role while drying at low temperature.

It should be noted that all non-starch and protein components produced by the production process enter a syrup production process in the step S5.

In some examples of the present disclosure, in S5, a ratio of a mass of the first precipitate to a mass of the water added during the washing of the first precipitate ranges from 1:3 to 1:10.

In some examples of the present disclosure, in S5, first wastewater is generated during the washing and centrifugal dewatering of the first precipitate, the first wastewater is recycled, and the recycled first wastewater is applied to the process of blending the first slurry in S1.

In some examples of the present disclosure, in S6, a ratio of a mass of the second precipitate to a mass of the pure water added to the second precipitate ranges from 1:3 to 1:10; and
the heating temperature is kept to be 60° C. or more, and the heat preservation is performed for 10 min or more.

In some examples of the present disclosure, in S7, second wastewater is generated during the filtration and dewatering of the material, the second wastewater is recycled, and the recycled second wastewater is applied to the process of washing the first precipitate with the water in S5.

It should be noted that washing wastewater of the second precipitate is defined as the second wastewater.

In some examples of the present disclosure, in S7, the material is filtered by using a diaphragm plate and frame filter press to obtain the filter cake.

Quantitative experiments in the following examples were performed in triplicate and data is a mean or mean±SD (Standard Deviation) of the triplicate experiments.

### Example 1

1000 kg of rice was cleaned and crushed to 60-mesh, then 1500 kg of water was added, slurry grinding was performed, and uniform stirring was performed to form a first slurry. The first slurry was homogenized by using a high pressure homogenizer under a homogenization pressure of 20 MPa at a temperature of being controlled to be less than 55° C. to form a second slurry.

High temperature alpha-amylase (alpha-amylase is endoglucosidase that acts randomly on alpha-1,4-glucosidic bonds inside a starch chain, and amylose degradation products are glucose, maltose, and maltotriose) was added into the second slurry.

The second slurry and the amylase were heated to 110° C. in a reaction vessel, and heat preservation was performed for 90 min so that the second slurry sufficiently reacted with the amylase to obtain a raw rice syrup liquid.

Next, the raw rice syrup liquid was subjected to centrifugal separation by using a horizontal spiral centrifuge to separately obtain a liquid-phase rice sugar liquid and a solid first precipitate.

The obtained liquid-phase rice sugar liquid was sequentially subjected to decoloration, ion exchange and evaporation to obtain a rice syrup with the composition maltodextrin.

The obtained first precipitate was washed with water, centrifugal separation was performed again by using a horizontal spiral centrifuge, and the first precipitate was dewatered to obtain a second precipitate.

Wastewater obtained after the centrifugal separation of the first precipitate was first wastewater which can be recycled for use in a water addition process for blending the first slurry.

Pure water was added into the second precipitate, wherein a mass ratio of the second precipitate to the pure water was 1:10, uniform stirring was performed, the stirred material was heated to 90° C., and heat preservation was performed for 30 min to obtain a solid-liquid mixed material.

The material was dewatered by using a diaphragm plate and frame filter press to obtain a filter cake.

Water obtained after the dewatering of the material was second wastewater which can be recycled for use in the process of washing the first precipitate with the water.

The filter cake obtained after the plate and frame filtration was subjected to airflow drying to obtain a protein powder having a protein content of more than 80%.

### Example 2

1000 kg of rice was cleaned and crushed to 60-mesh, then 1600 kg of water was added, slurry grinding was performed, and uniform stirring was performed to form a first slurry. The first slurry was homogenized by using a high pressure homogenizer under a homogenization pressure of 16 MPa at a temperature of being controlled to be less than 55° C. to form a second slurry.

Medium temperature alpha-amylase was added into the second slurry, and the second slurry and the amylase were heated to 50° C. in a reaction vessel, and heat preservation was performed for 80 min so that the second slurry sufficiently reacted with the amylase to obtain a raw rice syrup liquid.

Next, the raw rice syrup liquid was subjected to centrifugal separation by using a horizontal spiral centrifuge to separately obtain a liquid-phase rice sugar liquid and a solid first precipitate.

The obtained liquid-phase rice sugar liquid was sequentially subjected to decoloration, ion exchange and evaporation to obtain a rice syrup with the composition maltodextrin.

The obtained first precipitate was washed with water, centrifugal separation was performed again by using a horizontal spiral centrifuge, and the first precipitate was dewatered to obtain a second precipitate.

Wastewater obtained after the centrifugal separation of the first precipitate was first wastewater which can be recycled for use in a water addition process for blending the first slurry.

Pure water was added into the second precipitate, wherein a mass ratio of the second precipitate to the pure water was 1:3, uniform stirring was performed, the stirred material was heated to 70° C., and heat preservation was performed for 15 min to obtain a solid-liquid mixed material.

The material was dewatered by using a diaphragm plate and frame filter press to obtain a filter cake.

Water obtained after the dewatering of the material was second wastewater which can be recycled for use in the process of washing the first precipitate with the water.

The filter cake obtained after the plate and frame filtration was subjected to airflow drying to obtain a protein powder having a protein content of more than 80%.

### Example 3

2000 kg of rice was cleaned and crushed to 50-mesh, then 3200 kg of water was added, slurry grinding was performed, and uniform stirring was performed to form a first slurry. The first slurry was homogenized by using a high pressure homogenizer under a homogenization pressure of 20 MPa at a temperature of being controlled to be less than 55° C. to form a second slurry.

Medium temperature alpha-amylase was added into the second slurry, and the second slurry and the amylase were heated to 55° C. in a reaction vessel, and heat preservation was performed for 75 min so that the second slurry sufficiently reacted with the amylase to obtain a raw rice syrup liquid.

Next, the raw rice syrup liquid was subjected to centrifugal separation by using a horizontal spiral centrifuge to separately obtain a liquid-phase rice sugar liquid and a solid first precipitate.

The obtained liquid-phase rice sugar liquid was sequentially subjected to saccharification, decoloration, ion exchange and evaporation to obtain a rice syrup with the composition malt syrup.

The obtained first precipitate was washed with water, centrifugal separation was performed again by using a horizontal spiral centrifuge, and the first precipitate was dewatered to obtain a second precipitate.

Wastewater obtained after the centrifugal separation of the first precipitate was first wastewater which can be recycled for use in a water addition process for blending the first slurry.

Pure water was added into the second precipitate, wherein a mass ratio of the second precipitate to the pure water was 1:6, uniform stirring was performed, the stirred material was heated to 60° C., and heat preservation was performed for 20 min to obtain a solid-liquid mixed material.

The material was dewatered by using a plate and frame filter press to obtain a filter cake.

Water obtained after the dewatering of the material was second wastewater which can be recycled for use in the process of washing the first precipitate with the water.

The filter cake obtained after the plate and frame filtration was subjected to microwave vacuum drying at a drying temperature of 60° C. to obtain a protein powder having a protein content of more than 82%.

### Example 4

1200 kg of rice was cleaned and crushed to 65-mesh, then 2000 kg of water was added, slurry grinding was performed, and uniform stirring was performed to form a first slurry. The first slurry was homogenized by using a high pressure homogenizer under a homogenization pressure of 18 MPa at a temperature of being controlled to be less than 50° C. to form a second slurry.

High temperature alpha-amylase was added into the second slurry, and the second slurry and the amylase were heated to 105° C. in a reaction vessel, and heat preservation was performed for 85 min so that the second slurry sufficiently reacted with the amylase to obtain a raw rice syrup liquid.

Next, the raw rice syrup liquid was subjected to centrifugal separation by using a horizontal spiral centrifuge to separately obtain a liquid-phase rice sugar liquid and a solid first precipitate.

The obtained liquid-phase rice sugar liquid was sequentially subjected to decoloration, ion exchange and evaporation to obtain a rice syrup with the composition maltodextrin.

The obtained first precipitate was washed with water, centrifugal separation was performed again by using a horizontal spiral centrifuge, and the first precipitate was dewatered to obtain a second precipitate.

Wastewater obtained after the centrifugal separation of the first precipitate was first wastewater which can be recycled for use in a water addition process for blending the first slurry.

Pure water was added into the second precipitate, wherein a mass ratio of the second precipitate to the pure water was 1:5.5, uniform stirring was performed, the stirred material was heated to 85° C., and heat preservation was performed for 15 min to obtain a solid-liquid mixed material.

The material was dewatered by using a plate and frame filter press to obtain a filter cake.

Water obtained after the dewatering of the material was second wastewater which can be recycled for use in the process of washing the first precipitate with the water.

The filter cake obtained after the plate and frame filtration was subjected to airflow drying to obtain a protein powder having a protein content of more than 80%.

The above description is only the preferred embodiments of the present disclosure, and it should be noted that, for those of ordinary skill in the art, several improvements and substitutions can be made without departing from the technical principle of the present disclosure, and these improvements and substitutions should also be regarded as the protection scope of the present disclosure.

What is claimed is:

1. A method for producing a high-purity rice protein powder and a rice syrup, comprising the steps of:
S1, crushing rice, adding water for blending to obtain a first slurry, and homogenizing the first slurry with a high pressure homogenizer to obtain a second slurry;
S2, adding amylase into the second slurry, heating the resulting mixture, and performing heat preservation so that the second slurry sufficiently reacts with the amylase to obtain a raw rice syrup liquid;
S3, subjecting the raw rice syrup liquid to centrifugal separation to separately obtain a liquid-phase rice sugar liquid and a first precipitate;
S4, sequentially subjecting the liquid-phase rice sugar liquid obtained in S3 to decoloration, ion exchange, and evaporation to obtain the rice syrup;
S5, adding second wastewater into the first precipitate obtained in S3, performing washing and performing centrifugal dewatering to obtain a second precipitate;
S6, adding pure water into the second precipitate, performing uniform stirring, and performing heating and heat preservation to obtain a solid-liquid mixed material;
S7, subjecting the material to filtration and dewatering to obtain a filter cake and second wastewater; and
S8, crushing and drying the filter cake to obtain the high-purity rice protein powder.

2. The method for producing a high-purity rice protein powder and a rice syrup according to claim 1, wherein in S5, first wastewater is generated during the washing and centrifugal dewatering of the first precipitate, the first wastewater is recycled, and the recycled first wastewater is applied to the process of blending the first slurry in S1.

3. The method for producing a high-purity rice protein powder and a rice syrup according to claim 1, wherein in S7, second wastewater is generated during the filtration and dewatering of the material, the second wastewater is recycled, and the recycled second wastewater is applied to the process of washing the first precipitate with the water in S5.

4. The method for producing a high-purity rice protein powder and a rice syrup according to claim 1, wherein in S1, a mass ratio of the rice to the water in the first slurry is 1:1.5.

5. The method for producing a high-purity rice protein powder and a rice syrup according to claim 1, wherein in S1, the first slurry is homogenized under a homogenization pressure of greater than 15 MPa at a temperature of not more than 55° C.

6. The method for producing a high-purity rice protein powder and a rice syrup according to claim 1, wherein in S2, the amylase is alpha-amylase; the second slurry is heated at a temperature in the range of 50-110° C.; and a duration of the heat preservation during the reaction between the second slurry and the amylase is in the range of 60-120 min.

7. The method for producing a high-purity rice protein powder and a rice syrup according to claim 1, wherein the centrifugal separation in S3 uses a horizontal spiral centrifuge.

8. The method for producing a high-purity rice protein powder and a rice syrup according to claim 1, wherein in S5, a ratio of a mass of the first precipitate to a mass of the water added during the washing of the first precipitate ranges from 1:3 to 1:10.

9. The method for producing a high-purity rice protein powder and a rice syrup according to claim 1, wherein in S6, a ratio of a mass of the second precipitate to a mass of the pure water added to the second precipitate ranges from 1:3 to 1:10; and the heating temperature is kept to be 60° C. or more, and the heat preservation is performed for 10 min or more.

10. The method for producing a high-purity rice protein powder and a rice syrup according to claim 1, wherein in S7, the material is filtered by using a plate and frame filter press to obtain the filter cake; and the plate and frame filter press employs a diaphragm plate and frame filter press.

11. The method for producing a high-purity rice protein powder and a rice syrup according to claim 1, wherein in S8, a drying device is an airflow drying device or a vacuum microwave dryer.

12. The method for producing a high-purity rice protein powder and a rice syrup according to claim 1, wherein in S4, the liquid-phase rice sugar liquid can further be subjected to saccharification, activated carbon decoloration, ion exchange, evaporation and concentration to obtain the rice syrup.
